(19) United States
(12) Patent Application Publication
(10) Pub. No.: US 2008/0124795 A1
(43) Pub. Date: May 29, 2008

(54) Vascular Cell Culture Patterning Substrate

(76) Inventors: Hideyuki Miyake, Tokyo (JP); Hideshi Hattori, Tokyo (JP)

Correspondence Address: SEYFARTH SHAW LLP, 131 S. DEARBORN ST., SUITE 2400, CHICAGO, IL, 60603-5803, US

(21) Appl. No.: 10/592,199

(22) PCT Filed: Mar. 10, 2005
(86) PCT No.: PCT/JP05/04192

§ 371 Date: Sep. 8, 2006

(30) Foreign Application Priority Data
Mar. 10, 2004 (JP) 2004-067032

(51) Int. Cl. C12N 5/02

(52) U.S. Cl. 435/402

(57) ABSTRACT

A main object of the present invention is to provide a vascular cell culture patterning substrate, which can efficiently form a plurality of blood vessels on one substrate. To achieve the object, the present invention provides a vascular cell culture patterning substrate comprising: a base material; a vascular cell adhesion portion formed in at least two substantially parallel lines on the base material, and has an adhesive properties to a vascular cell which forms a blood vessel; and a vascular cell adhesion-inhibiting portion formed in between two adjacent vascular cell adhesion portions on the base material, and inhibits adhesion to the vascular cell,

wherein the vascular cell adhesion-inhibiting portions are formed at such a surface distance that, upon adhesion of the vascular cells to the vascular cell adhesion portions, the cells on the two vascular cell adhesion portions adjacent to the vascular cell adhesion-inhibiting portion are not contacted with one another via pseudopods.

### TECHNICAL FIELD

The present invention relates to a vascular cell culture patterning substrate, which is used for culturing vascular cells to form blood vessels.

### BACKGROUND ART

At present, cell cultures of various animals and plants are performed, and also new cell culture methods are in development. The technologies of the cell culture are utilized, such as to elucidate the biochemical phenomena and natures of cells and to produce useful substances. Furthermore, with cultured cells, an attempt to investigate the physiological activity and toxicity of artificially synthesized medicals is under way.

Some cells, particularly a lot of animal cells have the adhesion dependency of adhering to some materials and growing thereon, and cannot survive for a long period under a flotation condition out of organisms. For culturing cells having such adhesion dependency, a carrier to which cells can adhere is necessary, and in general, a plastic culture dish with uniformly applied cell adhesive proteins such as collagen, fibronectin and the like is used. It is known that these cell adhesive proteins act on cultured cells, make the cells adhere easily, and exert an influence on the form of cells.

On the other hand, there is a technology reported of adhering cultured cells only onto a small part on a base material and arranging them. By such a technology, it is made possible to apply cultured cells to artificial organs, biosensors, bioreactors and the like. As the method for arranging cultured cells, there is a method adopted in which a base material having a surface that forms a pattern different in easiness of adhesion to cells is used, cells are cultured on the surface of this base material and allowed to adhere only onto surfaces processed so that cells adhere, and thereby the cells are arranged.

For example, in the patent document 1, an electric charge-retaining medium on which an electrostatic pattern is formed is applied to culture cells for the purpose of proliferating nerve cells in a form of circuit, and the like. Furthermore, the patent document 2 tries to arrange cultured cells on a surface on which a cell adhesion-inhibiting or cell adhesive photosensitive hydrophilic polymer has been patterned by a photolithography method.

Furthermore, the patent document 3 discloses a cell culture base material on which a substance such as collagen and the like affecting on the adhesion ratio and form of cells is patterned, and a method for producing this base material by a photolithography method. By culturing cells on such a base material, a larger amount of cells can be adhered on a surface on which collagen or the like is patterned, to realize patterning of cells.

When vascular cells for forming blood vessels are cultured by employing such methods to form blood vessels, blood vessels will be formed by culturing vascular cells on vascular cell culture portions patterned in a line form. In this case, however, when a plurality of blood vessels is formed on one substrate, cell pseudopods will be extended between cells adhering to the adjacent vascular cell culture portions. Accordingly, there is a problem that when vascular tissues are regenerated by stimulating the vascular cells on the vascular cell culture portions, the formed vascular tissues will be adhered to one another via pseudopods contacting with adjacent vascular lines so as to form blood vessels in a form different from objective blood vessels, or blood vessels may be cut by stress upon such adhesion, thus failing to form objective blood vessels. To solve this problem, when forming only one blood vessel on one substrate, pseudopods will not be generated between vascular cells adhering to a line pattern, thereby causing no adhesion between blood vessels. However, there is a problem of low production efficiency.

### Patent Document 1: Japanese Patent Application Laid-Open (JP-A) No. 2-245181

### Patent Document 2: JP-A No. 3-7576

### Patent Document 3: JP-A No. 5-176753

### DISCLOSURE OF INVENTION

### Problem to be Solved by the Invention

Accordingly, it has been desired to provide a vascular cell culture patterning substrate, which can efficiently form a plurality of blood vessels on one substrate.

### Means for Solving the Problem

The present invention provides a vascular cell culture patterning substrate comprising: a base material; a vascular cell adhesion portion formed in at least two substantially parallel lines on the base material, and has adhesive properties to a vascular cell which forms a blood vessel; and a vascular cell adhesion-inhibiting portion formed in between two adjacent vascular cell adhesion portions on the base material, and inhibits adhesion to the vascular cell,

wherein the vascular cell adhesion-inhibiting portions are formed at such a surface distance that, upon adhesion of the vascular cells to the vascular cell adhesion portions, the cells on the two vascular cell adhesion portions adjacent to the vascular cell adhesion-inhibiting portion are not contacted with one another via pseudopods.

In the present invention, since the vascular cell adhesion-inhibiting portions are formed at the above-mentioned surface distances, when culturing the vascular cells by adhering to the vascular cell adhesion portions, the vascular cells on adjacent vascular cell adhesion portions will not be bound to one another so that the vascular cells will not be ruptured, thus enabling to culture the vascular cells in an objective shape.

In the invention described above, the vascular cell adhesion-inhibiting portion may be formed in a convex form. In this case, because of the height of the vascular cell adhesion-inhibiting portion, vascular cells adhering to the vascular cell adhesion portion can be prevented from being contacted with one another via pseudopods on the vascular cell adhesion-inhibiting portion. Therefore, the linear distance between the two vascular cell adhesion portions can be reduced. Further, the extension of pseudopods from cells cultured on a plane can be inhibited by the convex shielding material. Therefore, providing the convex shielding material is more effective than increasing the linear distance. According to such function, there is also an advantage that a larger number of blood vessels can be manufactured on one substrate by preventing pseudopods from extending and contacting with one another between the vascular lines.

Further, the present invention provides a method for manufacturing a blood vessel, wherein the above-mentioned vascular cell culture patterning substrate is used to culture a vascular cell.

In the present invention, by using the vascular cell culture patterning substrate, a high-quality blood vessel can be formed since the blood vessel will not be rupture while culturing the vascular cell.

### EFFECT OF THE INVENTION

According to the present invention, when culturing the vascular cell by adhering them on the vascular cell adhesion portion, the pseudopods generated from the blood vessels on the adjacent vascular cell adhesion portions will not contact to one another. As a result, the blood vessels will not be ruptured so that an effect, that the blood vessels can be formed in an objective shape, can be achieved.

### BRIEF DESCRIPTION OF DRAWINGS

FIG. 1 is a plan view showing an example of the vascular cell culture patterning substrate of the present invention.

FIG. 2 is a schematic sectional view showing an example of the vascular cell culture patterning substrate of the present invention.

FIG. 3 is a schematic sectional view showing an example of the photocatalyst-containing layer side substrate used in the present invention.

FIG. 4 is a schematic sectional view showing another example of the photocatalyst-containing layer side substrate used in the present invention.

FIG. 5 is a schematic sectional view showing another example of the photocatalyst-containing layer side substrate used in the present invention.

FIG. 6 is an illustration showing another example of the method for forming the vascular cell adhesion portion and vascular cell adhesion-inhibiting portion in the vascular cell culture patterning substrate of the present invention.

### DESCRIPTION OF SYMBOLS

1: Base material

2: Vascular cell adhesion portion

3: Vascular cell adhesion-inhibiting portion

### BEST MODE FOR CARRYING OUT THE INVENTION

The present invention relates to a vascular cell culture patterning substrate, which is used in culturing vascular cells to form blood vessels, as well as a method for manufacturing blood vessels by using the vascular cell culture patterning substrate. Hereinafter, these will be explained respectively.

### A. Vascular Cell Culture Patterning Substrate

First, a vascular cell culture patterning substrate of the present invention will be explained. The vascular cell culture patterning substrate of the present invention comprises: a base material; a vascular cell adhesion portion formed in at least two substantially parallel lines on the base material, and has adhesive properties to a vascular cell which forms a blood vessel; and a vascular cell adhesion-inhibiting portion formed in between two adjacent vascular cell adhesion portions on the base material, and inhibits adhesion to the vascular cell, wherein the vascular cell adhesion-inhibiting portions are formed at such a surface distance that, upon adhesion of the vascular cells to the vascular cell adhesion portions, the cells on the two vascular cell adhesion portions adjacent to the vascular cell adhesion-inhibiting portion are not contacted with one another via pseudopods.

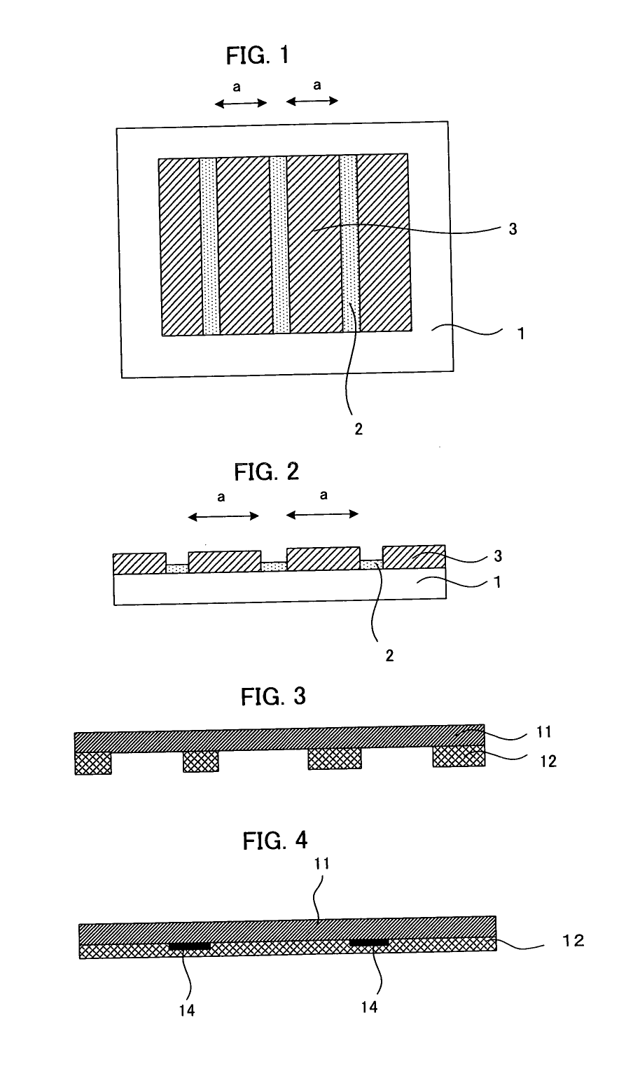

As shown in FIG. 1 for example, the vascular cell culture patterning substrate of the present invention comprises: a base material 1; vascular cell adhesion portions 2 formed on the base material 1, have adhesive properties to a vascular cell, and are formed as substantially parallel lines of at least two; and vascular cell adhesion-inhibiting portions 3 formed in between the vascular cell adhesion portions 2 and inhibit adhesion to vascular cells, wherein the vascular cell adhesion-inhibiting portions 3 are formed at such surface distances “a” that, upon adhesion of vascular cells to the adjacent vascular cell adhesion portions 2, the cells are not contacted with one another via pseudopods on the vascular cell adhesion-inhibiting portions 3.

In the present invention, the surface distance of the vascular cell adhesion-inhibiting portion is established such that vascular cells adhering to the adjacent vascular cell adhesion portions are not contacted with one another via pseudopods. Thus, the vascular cells can be cultured highly precisely along a pattern of the vascular cell adhesion portion. Accordingly, the cells can be prevented from being attached to one another between the vascular cell adhesion portions, and from the formed blood vessels being ruptured due to a stress on adjacent blood vessels. Therefore, a plurality of blood vessels can be formed efficiently on one substrate.

As shown in FIG. 2 for example, in the vascular cell culture patterning substrate of the present invention, convexoconcave may be formed on the substrate 1, such that the vascular cell adhesion portion-inhibiting portions 3 are a convex portion, while the vascular cell adhesion portions 2 are a concave portion. In this case, due to the height of the vascular cell adhesion-inhibiting portion 3, vascular cells adhering to the adjacent vascular cell adhesion portions 2 can be prevented from contacting with one another via pseudopods. Accordingly, the linear distance “a” between the adjacent vascular cell adhesion portions 2 can be made shorter by forming the vascular cell adhesion-inhibiting portions 3 in a convex form, compared to a case where the vascular cell adhesion-inhibiting portions 3 are formed in a flat form. Since extension of pseudopods from the cells cultured on the plane can be inhibited by the convex shielding materials, a higher effect can be achieved by providing the convex shielding material than by increasing the linear distance. Therefore, by preventing pseudopods from extending and contacting with one another between the vascular lines, a larger number of blood vessels can be manufactured on one substrate, which is preferable from the viewpoint of efficiency of manufacturing.

As used herein, the term “surface distance” refers to the distance between the adjacent vascular cell adhesion portions in the case where the vascular cell adhesion-inhibiting portion is flat. Moreover, in the case where the vascular cell adhesion-inhibiting portion is formed in a convexoconcave form, it refers to the distance along the surface of the vascular cell adhesion-inhibiting portion, that is, the distance connecting the adjacent vascular cell adhesion portions including the length of the side portions of the convexoconcave portion.

Hereinafter, the respective constitutions of the vascular cell culture patterning substrate of the present invention will be described in detail.

### (Vascular Cell Adhesion Portion)

First, the vascular cell adhesion portion of the vascular cell culture patterning substrate of the present invention is described. The vascular cell adhesion portion in the present invention is a region formed on a base material described later and has adhesive properties to a vascular cell for forming a blood vessel. In the present invention, at least two vascular cell adhesion portions are formed as substantially parallel lines on the vascular cell culture patterning substrate. As used herein, the term “substantially parallel” refers not only to completely parallel but also to substantially parallel, that is, the two lines are not crossed in a region. It includes, for example, lines such as zigzag lines that exist without crossing one another. The term “substantially parallel” also refers to portions that are not crossed in a crossed structure such as a net-like structure.

The shape of the vascular cell adhesion portion is not particularly limited insofar as it is formed in a line form. The shape is selected suitably depending on the shape of an objective blood vessel. Usually, the line width of the vascular cell adhesion portion shall be about 10 μm to 5000 μm, especially 20 μm to 100 μm, particularly 40 μm to 60 μm. A line width of less than 10 μm is not preferable because adhesion of vascular cells is made difficult. A line width of greater than 5000 μm, on the other hand, is not preferable either because almost all vascular cells will be adheres to the vascular cell adhesion portion in a spread state, thus making the cultured vascular cells hardly formable in the form of a blood vessel.

Concerning the phrase “vascular cell adhesion portion has adhesive properties to a vascular cell”, the adhesive properties to a vascular cell may be imparted by, for example, biochemical properties. Moreover, the adhesive properties to a vascular cell may be imparted by physicochemical properties.

Such vascular cell adhesion portion, for example, may be obtained by forming a vascular cell adhesion layer containing a vascular cell adhesive material having adhesive properties to a vascular cell. Moreover, for example, when a base material described later has adhesive properties to a vascular cell, the surface of the base material can be used as the vascular cell adhesion portion. The method for forming the vascular cell adhesion layer includes general printing methods, photolithographic techniques, and patterning methods using the action of a photocatalyst upon irradiation with energy.

A material having adhesive properties to a vascular cell which can be also used as a base material described later includes various kinds of glass, plasma-treated polystyrene, polypropylene etc. As the vascular cell adhesive material used in the vascular cell adhesion layer, cell adhesive materials used in general cell culture substrates etc. can be used. Examples of materials adhering to vascular cells for example by physicochemical properties include hydrophilic polystyrene, poly(N-isopropylacrylamide), basic polymers such as polylysine, basic compounds such as aminopropyltriethoxysilane, N-(2-aminoethyl)-3-aminopropyltrimethoxysilane etc., and condensates containing thereof. The vascular cell adhesive material having adhesive properties to a vascular cell biochemically includes fibronectin, laminine, tenascin, vitronectin, an RGD (arginine-glycine-aspartic acid) sequence-containing peptide, a YIGSR (tyrosine-isoleucine-glycine-serine-arginine) sequence-containing peptide, collagen, atelocollagen, gelatin, and mixtures thereof, for example matrigel etc.

In the present invention, particularly the vascular cell adhesion portion preferably has a vascular cell adhesion auxiliary portion in order to form an excellent blood vessel. The vascular cell adhesion auxiliary portion refers to a region not having adhesive properties to a vascular cell, which is formed in a fine pattern on the vascular cell adhesion portion. The vascular cell adhesion auxiliary portion is formed in such a fine pattern to an extent that, when vascular cells are adhered onto the vascular cell adhesion portion, binding of the vascular cells to one another in the vascular cell adhesion portion is not prevented. That is, to an extent that the vascular cells can be bound to one another even on the vascular cell adhesion auxiliary portion.

Generally, when vascular cells are adhered to a vascular cell culture region and cultured to form a tissue, the vascular cells are gradually arranged from the outside toward inside of a vascular cell adhesion portion. For forming a tissue, individual vascular cells should be changed morphologically and arranged, and this morphological change of the vascular cell also gradually occurs from the edge part toward center part of the vascular cell adhesion portion. Accordingly, when the width of the vascular cell adhesion portion is large, a tissue may not be formed in the center part of the vascular cell adhesion portion because of insufficient arrangement of the vascular cells, or the vascular cells may fail to adhere to the center part of the vascular cell adhesion portion. Moreover, the morphological change of the vascular cells in the center part of the vascular cell adhesion portion may be insufficient. Therefore, by forming the vascular cell adhesion auxiliary portion described above, the vascular cells can be arranged or morphologically changed from the edge part of the vascular cell adhesion auxiliary portion. Thereby, the vascular cells can be cultured without generating defects or inferior morphological change. Moreover, the vascular cell adhesion auxiliary portion is formed such that vascular cells adjacent to one another via the vascular cell adhesion auxiliary portion are not prevented from being adhered to one another. Thus, the width of the finally cultured vascular cells can be the same as the width of the vascular cell adhesion portion.

The vascular cell adhesion auxiliary portion is formed preferably in a line form in the vascular cell adhesion portion. The shape of the line is not particularly limited and can be in the form of, for example, a straight line, a curved line, a dotted line, a broken line, etc. The line width of the vascular cell adhesion auxiliary portion is preferably in the range of 0.5 μm to 10 μm, more preferably 1 μm to 5 μm. The width larger than the above range is not preferable because the vascular cells adjacent to one another via the vascular cell adhesion auxiliary portion will hardly interact with one another on the vascular cell adhesion auxiliary portion. When the width is smaller than the above range, on the other hand, the vascular cell adhesion auxiliary portion will be hardly formed by pattern forming techniques described later.

The vascular cell adhesion auxiliary portion may be formed to have a convexoconcave pattern (for example, zigzag etc.) in plane. The term “in plane” refers to the surface of a base material or a surface analogous thereto. The average distance from the edge part of the concave portion to the edge part of the convex portion, of the convexoconcave pattern, may be such a distance that when vascular cells are adhered to the vascular cell adhesion portion, the vascular cells are aligned in the same direction as the line direction of the vascular cell adhesion portion, and the average distance is particularly preferably in the range of 0.5 μm to 30 μm. The average distance from the edge part of the concave portion to the edge part of the convex portion of the convexoconcave pattern is determined by calculating the average of measured distances from the lowermost bottom to the uppermost top of each concavoconvex, within the range of 200 μm of the edge portion of the vascular cell adhesion auxiliary portion. Formation of the vascular cell adhesion auxiliary portion is same as the method for forming a vascular cell adhesion-inhibiting portion described below.

### (Vascular Cell Adhesion-Inhibiting Portion)

Now, the vascular cell adhesion-inhibiting portion in the present invention is described. The vascular cell adhesion-inhibiting portion in the present invention is not particularly limited as long as: it is formed in between the adjacent vascular cell adhesion portions on a base material described later; exhibits vascular cell adhesion-inhibiting properties of inhibiting adhesion to the vascular cells; and formed at such a surface distance that, upon adhesion of the vascular cells to the vascular cell adhesion portion, the vascular cells on the two vascular cell adhesion portions adjacent to the vascular cell adhesion-inhibiting portion are not contacted with one another via pseudopods. The vascular cell adhesion-inhibiting portion may be a flat region or may be formed in a convex form as described above.

When the vascular cell adhesion-inhibiting portion is a flat region, there is an advantage that the vascular cell adhesion-inhibiting portion can be easily formed. While, when the vascular cell adhesion-inhibiting region is formed in a convexoconcave form, since the height is included in the surface distance of the vascular cell adhesion-inhibiting portion, the linear distance between the adjacent vascular cell adhesion portions can be reduced. Therefore, a larger number of blood vessels can be formed on one substrate. Further, the extension of pseudopods from cells cultured on the plane can be inhibited by the convex shielding material so that, by providing the convex shielding material, higher effect can be achieved than by increasing the linear distance.

The surface distance of the vascular cell adhesion-inhibiting portion is suitably selected depending on the type of vascular cell adhering onto the vascular cell adhesion portion, the degree of vascular cell adhesion inhibition of the vascular cell adhesion-inhibiting portion, etc. Usually, it is about 200 μm to 600 μm, particularly about 300 μm to 500 μm. When the vascular cell adhesion-inhibiting portion is formed in a convex form, the height of the convexportion is usually preferably about 0.1 μm to 100 μm, more preferably about 1 μm to 10 μm. The “convex form” is not limited to a form whose section is a rectangular parallelepiped as shown in FIG. 2, and includes forms whose section is mountain-shaped, triangle, stairs-shaped, etc.

When the vascular cell adhesion-inhibiting portion is a flat region, the method for forming the vascular cell adhesion-inhibiting portion includes, for example, a method in which the vascular cell adhesion-inhibiting portion is formed by forming a vascular cell adhesion-inhibiting layer, having vascular cell adhesion-inhibiting properties of inhibiting adhesion to vascular cells, on a vascular cell adhesion-inhibiting portion. When a base material described later has vascular cell adhesion-inhibiting properties, the surface of the base material can be used as the vascular cell adhesion-inhibiting portion. The method for forming the vascular cell adhesion-inhibiting layer includes general printing methods, photolithographic techniques, and patterning methods using the action of a photocatalyst upon irradiation with energy.

When the vascular cell adhesion-inhibiting portion is formed in a convex form, it can be formed by, for example; attaching the material having vascular cell adhesion-inhibiting properties onto a base material described later; or forming a base material having vascular cell adhesion-inhibiting properties into a shape with a convex portion by molding.

The material having vascular cell adhesion-inhibiting properties which can be also used as a base material described later includes fluorine-based resins etc. such as polytetrafluoroethylene. As the vascular cell adhesion-inhibiting material used in the vascular cell adhesion-inhibiting layer, a cell adhesion-inhibiting material used in a general cell culture substrate etc. can be used, and for example, a material having high hydration ability can be used. When the material having high hydration ability is used, a hydration layer, wherein water molecules gather around the vascular cell adhesion-inhibiting material, is formed. Usually, such a material having high hydration ability has higher affinity for water molecules than for vascular cells. Thus, the vascular cells cannot be contacted to the material having high hydration ability so that the adhesive properties to the vascular cell will be low. The hydration ability refers to a property of hydrating with water molecules, and the high hydration ability is intended to mean that the material is easily hydrated with water molecules.

The material having high hydration ability which can be used as the vascular cell adhesion-inhibiting material includes, for example, polyethylene glycol, amphoteric ionic materials having a betaine structure, phospholipid-containing materials, etc.

As the vascular cell adhesion-inhibiting material, a material having water repellency or oil repellency can also be used. This is because, by the water repellency or oil repellency of the vascular cell adhesion-inhibiting material, the interaction such as hydrophobic interaction between vascular cells and the vascular cell adhesion-inhibiting material is made lower, thus decreasing adhesive properties to a vascular cell.

As the material having water repellency or oil repellency, for example, a material having a water-repellent or oil-repellant organic substituent can be used. Examples thereof include organopolysiloxanes exhibiting large strength obtained by hydrolyzing or polycondensating chloro- or alkoxysilanes by sol-gel reaction etc., as well as organopolysiloxanes obtained by crosslinking reactive silicone.

### (Base Material)

Now, the base material used in the present invention is described. The base material used in the present invention may be flat or may be formed such that the region serving as the vascular cell adhesion-inhibiting portion is in a convex form. As described above, the base material may have vascular cell adhesive properties or vascular cell adhesion-inhibiting properties.

As such base material, for example, an inorganic material such as metal, glass and silicon, or an organic material typified by plastics and the like can be used.

Moreover, plasticity, transparency, etc. of the base material is appropriately selected according to the types and uses of the cell culture patterning substrate.

### (Vascular Cell Culture Patterning Substrate)

The vascular cell culture patterning substrate of the present invention is not particularly limited insofar as it comprises a base material, the above-described vascular cell adhesion portion and the vascular cell adhesion-inhibiting portion, and if necessary, other suitable members etc. may be formed.

In the present invention, the vascular cell adhesion portion and vascular cell adhesion-inhibiting portion are preferably formed by using a layer whose adhesive properties to a vascular cell is changed by the action of a photocatalyst upon irradiation with energy. This is because the vascular cell adhesion portion and the vascular cell adhesion-inhibiting portion can be easily patterned.

The patterning substrate on which the vascular cell adhesion portion and the vascular cell adhesion-inhibiting portion are formed by the action of a photocatalyst upon irradiation with energy is described below. There are two embodiments for this patterning substrate. Hereinafter, the respective embodiments will be described separately in detail.

### First Embodiment

The first embodiment is a patterning substrate wherein: a vascular cell adhesion layer, containing a vascular cell adhesive material having at least adhesive properties to a vascular cell and is decomposed or denatured by the action of a photocatalyst upon irradiation with energy, is formed; and in the vascular cell adhesion-inhibiting portion, the vascular cell adhesive material is decomposed or denatured by the action of a photocatalyst upon irradiation with energy.

In this embodiment, for example, by arranging the vascular cell adhesion layer and a photocatalyst-containing layer side substrate comprising a photocatalyst-containing layer containing a photocatalyst so as to be opposite to each other and irradiating with energy in a pattern of a vascular cell adhesion-inhibiting portion to be formed, the vascular cell adhesive material in the vascular cell adhesion layer will be decomposed or denatured by the action of the photocatalyst in the photocatalyst-containing layer to form a vascular cell adhesion-inhibiting portion.

In this embodiment, there is an advantage that, when vascular cells are adhered to the vascular cell adhesion portion on the vascular cell culture patterning substrate to manufacture blood vessels, by irradiating the vascular cell adhesion-inhibiting portion forming region with energy by using the photocatalyst-containing layer, the vascular cells adhered to the vascular cell adhesion-inhibiting portion can be removed by the action of the photocatalyst, and thus the vascular cells cultured in a highly precise pattern can be maintained.

In this embodiment, the surface distance of the vascular cell adhesion-inhibiting portion is usually about 200 μm to 600 μm, particularly about 300 μm to 500 μm. In this range, the vascular cells can be prevented from contacting with each other via pseudopods between the adjacent vascular cell adhesion portions.

Hereinafter, the vascular cell adhesion layer, photocatalyst-containing layer side substrate and the method for forming the vascular cell adhesion-inhibiting portion by using the photocatalyst-containing layer side substrate, used in this embodiment, are described.

a. Vascular Cell Adhesion Layer

Now, the vascular cell adhesion layer used in this embodiment is described. The vascular cell adhesion layer used in this embodiment is a layer having at least a vascular cell adhesive material having adhesive properties to a vascular cell. Generally, a layer used as a layer having adhesive properties to a vascular cell can be used.

The type etc. of the vascular cell adhesive material contained in the vascular cell adhesion layer in this embodiment are not particularly limited insofar as the material has adhesive properties to a vascular cell and is decomposed or denatured by the action of the photocatalyst upon irradiation with energy. Here, “having adhesive properties to a vascular cell” means being good in adhesion to a vascular cell. For instance, when the adhesive properties to a vascular cell differ depending on the kind of vascular cells, it means to be good in the adhesion with target vascular cells.

The vascular cell adhesive material used in the present embodiment has such adhesive properties to a vascular cell. Those losing the adhesive properties to a vascular cell or those changed into ones having the vascular cell adhesion-inhibiting properties of inhibiting adhesion to vascular cells, by being decomposed or denatured by the action of the photocatalyst upon irradiation with energy, are used.

As such materials having the adhesive properties to a vascular cell, there are two kinds. One being materials having the adhesive properties to a vascular cell owing to physicochemical characteristics and the other being materials having the adhesive properties to a vascular cell owing to biochemical characteristics.

As physicochemical factors that determine the adhesive properties to a vascular cell of the materials having the adhesive properties to a vascular cell owing to the physicochemical characteristics, the surface free energy, the electrostatic interaction and the like can be cited. For instance, when the adhesive properties to a vascular cell is determined by the surface free energy of the material, if the material has the surface free energy in a predetermined range, the adhesive properties between the vascular cells and the material becomes good. If it deviates from the predetermined range the adhesive properties between the vascular cells and material is deteriorated. As such changes of the adhesive properties to a vascular cell due to the surface free energy, experimental results shown in Data, for instance, CMC Publishing Co., Ltd. “Biomaterial no Saisentan”, Yoshito IKADA (editor), p. 109, lower part are known. As materials having the adhesive properties to a vascular cell owing to such a factor, for instance, hydrophilic polystyrene, poly (N-isopropyl acrylamide) and the like can be cited. When such a material is used, by the action of the photocatalyst upon irradiation with energy, for instance, a functional group on a surface of the material is substituted, decomposed or the like to cause a change in the surface free energy, resulting in one that does not have the adhesive properties to a vascular cell or one that has the vascular cell adhesion-inhibiting properties.

When the adhesive properties between vascular cells and a material is determined owing to the electrostatic interaction or the like, for instance, the adhesive properties to a vascular cell are determined by an amount of positive electric charges and the like that the material has. As materials having the adhesive properties to a vascular cell owing to such electrostatic interaction, basic polymers such as polylysine; basic compounds such as aminopropyltriethoxysilane, N-(2-aminoethyl)-3-aminopropyltrimethoxysilane; and condensates and the like including these can be cited. When such materials are used, by the action of the photocatalyst upon irradiation with energy, the above-mentioned materials are decomposed or denatured. Thereby, for instance, an amount of positive electric charges present on a surface can be altered, resulting in one that does not have the adhesive properties to a vascular cell or one that has the vascular cell adhesion-inhibiting properties.

As materials having the adhesive properties to a vascular cell owing to the biological characteristics, ones that are good in the adhesive properties with particular vascular cells or ones that are good in the adhesive properties with many vascular cells can be cited. Specifically, fibronectin, laminin, tenascin, vitronectin, RGD (arginine-glycine-asparagine acid) sequence containing peptide, YIGSR (tyrosine-isoleucine-glycine-serine-arginine) sequence containing peptide, collagen, atelocollagen, gelatin and mixture thereof, such as matrigel and the like, can be cited. When such materials are used, by the action of the photocatalyst upon irradiation with energy, for instance, a structure of the material is partially destroyed, or a principal chain is destroyed or the like, resulting in one that does not have the adhesive properties to a vascular cell or one that has the vascular cell adhesion-inhibiting properties.

Such a vascular cell adhesive material, though it differs depending on the kind of the materials and the like, is comprised in the vascular cell adhesion layer normally in the range of 0.01% by weight to 95% by weight, and preferably in the range of 1% by weight to 10% by weight. Thereby, a region that contains the vascular cell adhesive material can be made a region good in the adhesive properties to a vascular cell.

In this embodiment, not only the vascular cell adhesive material but also a binder etc. for improving strength, resistance etc. may be contained as necessity in the vascular cell adhesion layer. In the present embodiment, particularly as the binder, a material that, at least after the energy irradiation, has the vascular cell adhesion-inhibiting properties of inhibiting adhesion to vascular cells is preferably used. This is because the adhesion between vascular cells and the vascular cell adhesion-inhibiting portion, which is a region irradiated with energy, can thereby be reduced. As such a material, one that has the vascular cell adhesion-inhibiting properties prior to the energy irradiation or one that obtains the vascular cell adhesion-inhibiting properties by the action of the photocatalyst upon irradiation with energy may be used.

In the present embodiment, a material that becomes to have the vascular cell adhesion-inhibiting properties, particularly by the action of the photocatalyst upon irradiation with energy, is preferably used as a binder. Thereby, in a region prior to the energy irradiation, the adhesiveness between the vascular cell adhesive material and vascular cells is not inhibited, and only a region where energy is irradiated can be lowered in the adhesive properties to a vascular cell.

As materials that can be used as such a binder, for instance, ones in which a main skeleton has such a high bond energy, that cannot be decomposed by the photo-excitation of the photocatalyst, and has an organic substituent which can be decomposed by an action of the photocatalyst are preferably used. For instance, (1) organopolysiloxane that exhibits large strength by hydrolyzing or polycondensating chloro- or alkoxysilane or the like owing to a sol-gel reaction and the like, and (2) organopolysiloxane and the like in which reactive silicones excellent in the water repellency or oil repellency are crosslinked can be cited.

In the case of the (1), it is preferable to be organopolysiloxanes that are hydrolysis condensates or cohydrolysis condensates of at least one kind of silicon compounds expressed by a general formula:

YₙSiX₍₄₋ₙ₎

(Here, Y denotes an alkyl group, fluoroalkyl group, vinyl group, amino group, phenyl group, epoxy group or organic group containing the above, and X denotes an alkoxyl group, acetyl group or halogen. “n” is an integer of 0 to 3.). The number of carbons of the organic group expressed with Y is preferably in the range of 1 to 20, and the alkoxy group shown with X is preferably a methoxy group, ethoxy group, propoxy group or butoxy group.

As the reactive silicone according to the (2), compounds having a skeleton expressed by a general formula below can be cited.

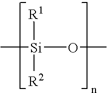

In the above general formula, n denotes an integer of 2 or more, R¹ and R² each represents a substituted or nonsubstituted alkyl group, alkenyl group, aryl group or cyanoalkyl group having 1 to 20 carbons, and a vinyl, phenyl and halogenated phenyl occupy 40% or less by mole ratio to a total mole. Furthermore, one in which R¹ and R²is a methyl group is preferable because the surface energy is the lowest, and a methyl group is preferably contained 60% or more by mole ratio. Still furthermore, a chain terminal or side chain has at least one or more reactive group such as a hydroxyl group in a molecular chain. When the material such as mentioned above is used, by the action of the photocatalyst upon irradiation with energy, a surface of an energy-irradiated region can be made high in the hydrophilicity. Thereby, the adhesion with vascular cells is inhibited, and the region where energy is irradiated can be made into a region on which the vascular cells do not adhere.

Together with the organopolysiloxanes, a stable organo silicium compound that does not cause a crosslinking reaction, such as dimethylpolysiloxanes, may be blended with a binder.

When the above-mentioned material is used as the vascular cell adhesion-inhibiting material, the contact angle thereof with water is preferably in the range of 15° to 120°, more preferably 20° to 100° before the material is irradiated with energy. According to this, the adhesion of the vascular cell adhesive material to vascular cells is not inhibited.

In the case of irradiating this vascular cell adhesion-inhibiting material with energy, it is preferred that the contact angle thereof with water becomes 100 or less. This range makes it possible to render the material having a high hydrophilicity and low adhesive properties to a vascular cell.

The contact angle with water referred to herein is a result obtained by using a contact angle measuring device (CA-Z model, manufactured by Kyowa Interface Science Co., Ltd.) to measure the contact angle of the material with water or a liquid having a contact angle equivalent to that of water (after 30 seconds from the time when droplets of the liquid are dropped down from its micro syringe), or a value obtained from a graph prepared from the result.

In the present embodiment, a decomposition substance or the like that causes such as a change in the wettability of a region where energy is irradiated, thereby lowers the adhesive properties to a vascular cell or that aides such a change may be contained.

As such decomposition substances, for instance, surfactants or the like that are decomposed and the like, by the action of the photocatalyst upon irradiation with energy, to be hydrophilic and the like to result in lowering the adhesive properties to a vascular cell can be cited. Specifically, nonionic surfactants: hydrocarbon based such as respective series of NIKKOL BL, BC, BO, and BB manufactured by Nikko Chemicals Co., Ltd.; and silicone based such as ZONYL FSN and FSO manufacture by Du Pont Kabushiki Kaisha, Surflon S-141 and 145 manufactured by ASAHI GLASS CO., LTD., Megaface F-141 and 144 manufactured by DAINIPPON INK AND CHEMICALS, Inc., FTERGENT F-200 and F-251 manufactured by NEOS, UNIDYNE DS-401 and 402 manufactured by DAIKIN INDUSTRIES, Ltd., and Fluorad FC-170 and 176 manufactured by 3M can be cited. Cationic surfactants, anionic surfactants and amphoteric surfactants also can be used.

Other than the surfactants, oligomers and polymers such as polyvinyl alcohol, unsaturated polyester, acrylic resin, polyethylene, diallyl phthalate, ethylene propylene diene monomer, epoxy resin, phenol resin, polyurethane, melamine resin, polycarbonate, polyvinyl chloride, polyamide, polyimide, styrene-butadiene rubber, chloroprene rubber, polypropylene, polybutylene, polystyrene, polyvinyl acetate, nylon, polyester, polybutadiene, polybenzimidazole, polyacrylonitrile, epichlorohydrine, polysulfide, polyisoprene and the like can be cited.

In the present embodiment, such a binder can be preferably comprised in the vascular cell adhesion layer, in the range of 5% by weight to 95% by weight, more preferably 40% by weight to 90% by weight, and particularly preferably 60% by weight to 80% by weight.

b. Photocatalyst-Containing Layer Side Substrate

First, the photocatalyst-containing layer side substrate, comprising a photocatalyst-containing layer containing a photocatalyst, used in this embodiment is described. The photocatalyst-containing layer side substrate used in this embodiment usually comprises a photocatalyst-containing layer containing a photocatalyst, which usually comprises a base body and a photocatalyst-containing layer formed on the base body. This photocatalyst-containing layer side substrate may have, for example, photocatalyst-containing layer side light-shielding portion formed in a pattern form, a primer layer, or the like. The following will describe each of the constituents of the photocatalyst-containing layer side substrate used in this embodiment.

(i) Photocatalyst-Containing Layer

First, the photocatalyst-containing layer used in the photocatalyst-containing layer side substrate is described. The photocatalyst-containing layer used in this embodiment is not particularly limited insofar as the layer is constituted such that the photocatalyst in the photocatalyst-containing layer can cause the decomposition or denaturation of the vascular cell adhesive material in the adjacent vascular cell adhesion layer. The photocatalyst-containing layer may be composed of a photocatalyst and a binder or may be made of a photocatalyst only. The property of the surface thereof may be lyophilic or repellent to liquid.

The photocatalyst-containing layer used in this embodiment may be formed on the whole surface of a base body, or as shown in, for example, FIG. 3, a photocatalyst-containing layer 12 may be formed in a pattern form on a base body 11.

As the photocatalyst that can be used in the present embodiment, specifically, for instance, titaniumdioxide (TiO₂), zinc oxide (ZnO), tin oxide (SnO₂), strontium titanate (SrTiO₃), tungsten oxide (WO₃), bismuth oxide (Bi₂O₃) andiron oxide (Fe₂O₃) that are known as photo-semiconductors can be cited. These can be used singularly or in combination of at least two kinds.

In the present embodiment, in particular, titanium dioxide, owing to a large band gap, chemical stability, non-toxicity, and easy availability, can be preferably used. There are two types of titanium dioxide, anatase type and rutile type, and both can be used in the present embodiment; however, the anatase type titanium dioxide is more preferable. An excitation wavelength of the anatase type titanium dioxide is 380 nm or less.

As such anatase type titanium dioxide, for instance, an anatase titania sol of hydrochloric acid deflocculation type (trade name: STS-02, manufactured by Ishihara Sangyo Kaisha, Ltd., average particle diameter: 7 nm, and trade name: ST-KO1, manufactured by Ishihara Sangyo Kaisha, Ltd.), an anatase titania sol of nitric acid deflocculation type (trade name: TA-15, manufactured by Nissan Chemical Industries Ltd., average particle diameter: 12 nm) and the like can be cited.

The smaller is a particle diameter of the photocatalyst, the better, because a photocatalyst reaction is caused more effectively. It is preferable to use the photocatalyst with an average particle diameter of 50 nm or less, and one having an average particle diameter of 20 nm or less can be particularly preferably used.

The photocatalyst-containing layer in this embodiment may be made of a photocatalyst only as described above or may be formed from a mixture with a binder.

The photocatalyst-containing layer consisting of a photocatalyst only is advantageous in costs because the efficiency of decomposing or denaturing the vascular cell adhesive material in the vascular cell adhesion layer is improved to reduce the treatment time. On the other hand, use of the photocatalyst-containing layer comprising a photocatalyst and a binder is advantageous in that the photocatalyst-containing layer can be easily formed.

An example of the method for forming the photocatalyst-containing layer made only of a photocatalyst may be a vacuum film-forming method such as sputtering, CVD or vacuum vapor deposition. The formation of the photocatalyst-containing layer by the vacuum film-forming method makes it possible to render the layer a homogeneous photocatalyst-containing layer made only of a photocatalyst. Thereby, the vascular cell adhesive material can be decomposed or denatured homogeneously. At the same time, since the layer is made only of a photocatalyst, the vascular cell adhesive material can be decomposed or denatured more effectively, as compared with the case of using a binder.

Another example of the method for forming the photocatalyst-containing layer made only of a photocatalyst, is the following method: for example, in the case that the photocatalyst is titanium dioxide, amorphous titania is formed on the base material, and then, calcinating so as to phase-change the titania to crystalline titania. The amorphous titania used in this case can be obtained, for example, by hydrolysis or dehydration condensation of an inorganic salt of titanium, such as titanium tetrachloride or titanium sulfate, or hydrolysis or dehydration condensation of an organic titanium compound, such as tetraethoxytitanium, tetraisopropoxytitanium, tetra-n-propoxytitanium, tetrabutoxytitanium or tetramethoxytitanium, in the presence of an acid. Next, the resultant is calcinated at 400° C. to 500° C. so as to be denatured to anatase type titania, and calcinated at 600° C. to 700° C. so as to be denatured to rutile type titania.

In the case of using a binder, the binder preferably having a high bonding energy, wherein its main skeleton is not decomposed by photoexcitation of the photocatalyst. Examples of such a binder include the organopolysiloxanes described in the above-mentioned item “Vascular cell adhesion layer”.

In the case of using such an organopolysiloxane as the binder, the photocatalyst-containing layer can be formed by dispersing a photocatalyst, the organopolysiloxane as the binder, and optional additives if needed into a solvent to prepare a coating solution, and coating this coating solution onto the base material. The used solvent is preferably an alcoholic based organic solvent such as ethanol or isopropanol. The coating can be performed by a known coating method such as spin coating, spray coating, dip coating, roll coating, or bead coating. When the coating solution contains an ultraviolet curable component as the binder, the photocatalyst-containing layer can be formed by curing the coating solution through the irradiation of ultraviolet rays.

As the binder, an amorphous silica precursor can be used. This amorphous silica precursor is preferably a silicon compound represented by the general formula SiX₄, wherein X are a halogen, a methoxy group, an ethoxy group, an acetyl group or the like; a silanol which isahydrolyzate thereof; or a polysiloxane having an average molecular weight of 3000 or less.

Specific examples thereof include such as tetraethoxysilane, tetraisopropoxysilane, tetra-n-propoxysilane, tetrabutoxysilane, and tetramethoxysilane. In this case, the photocatalyst-containing layer can be formed by dispersing the amorphous silica precursor and particles of a photocatalyst homogeneously into a non-aqueous solvent, hydrolyzing with water content in the air to form a silanol onto a transparent base material, and then subjecting to dehydration polycondensation at room temperature. When the dehydration polycondensation of the silanol is performed at 100° C. or higher, the polymerization degree of the silanol increases so that the strength of the film surface can be improved. A single kind or two or more kinds of this binding agent may be used.

The content of the photocatalyst in the photocatalyst-containing layer can be set in the range of 5 to 60% by weight, preferably in the range of 20 to 40% by weight. The thickness of the photocatalyst-containing layer is preferably in the range of 0.05 to 10 μm.

Besides the above-mentioned photocatalyst and binder, the surfactant and so on used in the above-mentioned vascular cell adhesion layer can be incorporated into the photocatalyst-containing layer.

(ii) Base Body

The following will describe the base body used in the photocatalyst-containing layer side substrate. Usually, the photocatalyst-containing layer side substrate comprises at least a base body and a photocatalyst-containing layer formed on the base body. In this case, the material which constitutes the base body to be used is appropriately selected depending on the direction of energy irradiation which will be detailed later, necessity of the resulting pattern-forming body to be transparency, or other factors.

The base body used in this embodiment may be a member having flexibility, such as a resin film, or may be a member having no flexibility, such as a glass substrate. This is appropriately selected depending on the method of the energy irradiation.

An anchor layer may be formed on the base body in order to improve the adhesion between the surface of the base body and the photocatalyst-containing layer. The anchor layer may be made of, for example, a silane based or titanium based coupling agent.

(iii) Photocatalyst-Containing Layer Side Light-Shielding Portion

The photocatalyst-containing layer side substrate used in this embodiment may be a photocatalyst-containing layer side substrate on which photocatalyst-containing layer side light-shielding portion is formed in a pattern. When the photocatalyst-containing layer side substrate having photocatalyst-containing layer side light-shielding portion is used in this way, at the time of irradiating energy, it is not necessary to use any photomask or to carry out drawing irradiation with a laser light. Since alignment of the photomask and the photocatalyst-containing layer side substrate is not necessary, process can be made simple. Further, since expensive device for drawing irradiation is also not necessary, it is advantageous in costs.

Such a photocatalyst-containing layer side substrate having photocatalyst-containing layer side light-shielding portion can be classified into the following two embodiments, depending on the position where the photocatalyst-containing layer side light-shielding portion is formed.

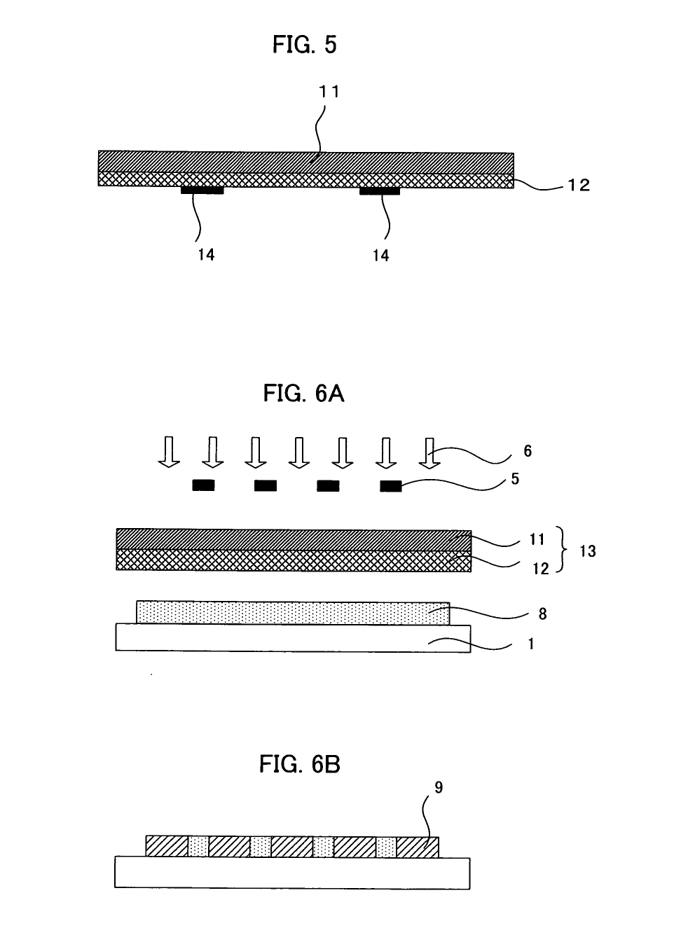

One of them is an embodiment, as shown in FIG. 4 for example, wherein photocatalyst-containing layer side light-shielding portion 14 is formed on a base body 11, and a photocatalyst-containing layer 12 is formed on the photocatalyst-containing layer side light-shielding portion 14 to obtain the photocatalyst-containing layer side substrate. The other example is an embodiment, as shown in FIG. 5 for example, wherein a photocatalyst-containing layer 12 is formed on a base body 11, and photocatalyst-containing layer side light-shielding portion 14 is formed thereon to obtain the photocatalyst-containing layer side substrate.

In any one of the embodiments, since the photocatalyst-containing layer side light-shielding portion is arranged near the region where the photocatalyst-containing layer and the vascular cell adhesion layer are arranged, the effect of energy-scattering in the base body or the like can be made smaller than in the case of using a photomask. Accordingly, irradiation of energy in a pattern can be more precisely attained.

In this embodiment, in the case of the embodiment wherein the photocatalyst-containing layer side light-shielding portion 14 is formed on a photocatalyst-containing layer 12 as shown in FIG. 5, there is an advantage that at the time of arranging the photocatalyst-containing layer and the vascular cell adhesion layer in a predetermined position, the photocatalyst-containing layer side light-shielding portion can be used as a spacer for making the interval constant, by making the film thickness of the photocatalyst-containing layer side light-shielding portion consistent with the width of the interval between the two layers.

In other words, when the photocatalyst-containing layer and the vascular cell adhesion layer are arranged so as to be facing each other at a predetermined interval, by arranging the photocatalyst-containing layer side light-shielding portion and the vascular cell adhesion layer in close contact to each other, the dimension of the predetermined interval can be made precise. When energy is irradiated in this state, vascular cell adhesion-inhibiting portion can be formed with a good precision since vascular cell adhesive material in the vascular cell adhesion layer, inside the region where the vascular cell adhesion layer and the light-shielding materials are in contact, is not decomposed or denatured.

The method for forming such photocatalyst-containing layer side light-shielding portion is not particularly limited, and may be appropriately selected in accordance with the property of the surface on which the photocatalyst-containing layer side light-shielding portion is to be formed, shielding ability against the required energy, and others. The light-shielding portion may be the same as those generally used. Thus, the detailed description thereof is omitted herein.

The above has described two cases, wherein the photocatalyst-containing layer side light-shielding portion is formed in between the base body and the photocatalyst-containing layer and is formed on the surface of the photocatalyst-containing layer. Besides, the photocatalyst-containing layer side light-shielding portion may be formed on the base body surface of the side on which the photocatalyst-containing layer is not formed. In this embodiment, for example, a photomask can be made in close contact to this surface to such a degree that the photomask in removable. Thus, this embodiment can be preferably used for the case that the pattern of the vascular cell adhesion auxiliary portions is changed for every small lot.

(iv) Primer Layer

The following will describe a primer layer used in the photocatalyst-containing layer side substrate of this embodiment. In this embodiment, when photocatalyst-containing layer side light-shielding portion is formed into a pattern on a base body and a photocatalyst-containing layer is formed thereon so as to prepare a photocatalyst-containing layer side substrate described above, a primer layer may be formed between the photocatalyst-containing layer side light-shielding portion and the photocatalyst-containing layer.

The effect and function of this primer layer are not necessarily clear, but would be as follows: by forming the primer layer between the photocatalyst-containing layer side light-shielding portion and the photocatalyst-containing layer, the primer layer is assumed to exhibit a function of preventing the diffusion of impurities from the photocatalyst-containing layer side light-shielding portion and openings present between the photocatalyst-containing layer side light-shielding portions, in particular, residues generated when the photocatalyst-containing layer side light-shielding portion is patterned, or metal or metal ion impurities; the impurities being factors for blocking the decomposition or denaturation of the vascular cell adhesive material by action of the photocatalyst. Accordingly, by forming the primer layer, it is possible to process the decomposition or denaturation of the vascular cell-adhesive material with high sensitivity, so as to yield vascular cell adhesion-inhibiting portion which are highly precisely formed.

The primer layer in this embodiment is a layer for preventing the effect of the photocatalyst from being affected by the impurities present in not only the photocatalyst-containing layer side light-shielding portion but also in the openings formed between the photocatalyst-containing layer side light-shielding portions. It is therefore preferred to form the primer layer over the entire surface of the photocatalyst-containing layer side light-shielding portion including the openings.

The primer layer in this embodiment is not particularly limited insofar as the primer layer is formed not to bring the photocatalyst-containing layer side light-shielding portion and the photocatalyst-containing layer of the photocatalyst-containing layer side substrate into contact with each other.

A material that forms the primer layer, though not particularly limited, is preferably an inorganic material that is not likely to be decomposed by the action of the photocatalyst. Specifically, amorphous silica can be cited. When such amorphous silica is used, a precursor of the amorphous silica is preferably a silicon compound that is represented by a general formula, SiX₄, wherein X being halogen, methoxy group, ethoxy group, acetyl group or the like; silanol that is a hydrolysate thereof, or polysiloxane having an average molecular weight of 3000 or less.

A film thickness of the primer layer is preferably in the range of 0.001 μm to 1 μm and particularly preferably in the range of 0.001 μm to 0.1 μm.

c. Method for Forming Vascular Cell Adhesion-Inhibiting Portion

Hereinafter, the method for forming the vascular cell adhesion-inhibiting portion in this embodiment is described. In this embodiment, for example as shown in FIG. 6, a vascular cell adhesion layer 8 formed on a base material 1, and a photocatalyst-containing layer 12 of a photocatalyst-containing layer side substrate 13, are arranged with a predetermined space and irradiated with energy 6 from a predetermined direction, for example, via photomask 5 or the like (FIG. 6(a)). The vascular cell adhesive material in the region irradiated with energy is thereby decomposed or denatured, thus forming the vascular cell adhesion-inhibiting portion 9 having no adhesive properties to a vascular cell (FIG. 6(b)). In this case, when the vascular cell adhesive material is decomposed for example by the action of a photocatalyst upon irradiation with energy, the vascular cell adhesion-inhibiting portion contains a small amount of the vascular cell adhesive material or decomposed products of the vascular cell adhesive material. Otherwise, the vascular cell adhesion layer is completely decomposed and removed to expose the base material. When the vascular cell adhesive material is denatured by the action of a photocatalyst upon irradiation with energy, its denatured products are contained in the vascular cell adhesion-inhibiting portion.

The above-mentioned wording “arranging” means that the layers are arranged in the state that the action of the photocatalyst can substantially work to the surface of the vascular cell adhesion layer, and include not only the state that the two layers actually contact each other, but also the state that the photocatalyst-containing layer and the vascular cell adhesion layer are arranged at a predetermined interval. The dimension of the interval is preferably 200 μm or less.

In this embodiment, the dimension of the above-mentioned interval is more preferably in the range of 0.2 μm to 10 μm, even more preferably in the range of 1 μm to 5 μm, since the precision of the pattern to be obtained becomes very good and further the sensitivity of the photocatalyst becomes high so as to make good efficiency of the decomposition or denaturation of the vascular cell adhesive material in the vascular cell adhesion layer. This range of the interval dimension is particularly effective for the vascular cell adhesion layer which is small in area, wherein the interval dimension can be controlled with a high precision.

Meanwhile, in the case of treating the vascular cell adhesion layer having large area, for example, 300 mm×300 mm or more in size, it is very difficult to make a fine interval as described above between the photocatalyst-containing layer side substrate and the vascular cell adhesion layer without contacting each other. Accordingly, when the vascular cell adhesion layer has a relatively large area, the interval dimension is preferably in the range of 10 to 100 μm, more preferably in the range of 50 to 75 μm. By setting the interval dimension in the above range, problems will not occur that: deterioration of patterning precision, such as blurring of the pattern; or the sensitivity of the photocatalyst deteriorates so that the efficiency of decomposing or denaturing the vascular cell adhesive material is also deteriorated. Further, there is an advantageous effect that the vascular cell adhesive material is not unevenly decomposed or denatured.

When energy is irradiated onto the vascular cell adhesion layer having a relatively large area as described above, the dimension of the interval, in a unit for positioning the photocatalyst-containing layer side substrate and the vascular cell adhesion layer inside the energy irradiating device, is preferably set in the range of 10 μm to 200 μm, more preferably in the range of 25 μm to 75 μm. The setting of the interval dimension value into this range makes it possible to arrange the photocatalyst-containing layer side substrate and the vascular cell adhesion layer without causing a large deterioration of patterning precision or of sensitivity of the photocatalyst, or bringing the substrate and the layer into contact with each other.

When the photocatalyst-containing layer and the surface of the vascular cell adhesion layer are arranged at a predetermined interval as described above, active oxygen species generated from oxygen and water by action of the photocatalyst can easily be released. In other words, if the interval between the photocatalyst-containing layer and the vascular cell adhesion layer is made narrower than the above-mentioned range, the active oxygen species are not easily released, so as to make the rate for decomposing or denaturing the vascular cell adhesive material unfavorably small. If the two layers are arranged at an interval larger than the above-mentioned range, the generated active oxygen species do not reach the vascular cell adhesion layer easily. In this case also, the rate for decomposing or denaturing the vascular cell adhesive material becomes unfavorably small.

The method for arranging the photocatalyst-containing layer and the vascular cell adhesion layer to make such a very small interval evenly therebetween is, for example, a method of using spacers. The use of the spacers in this way makes it possible to make an even interval. At the same time, the action of the photocatalyst does not work onto the surface of the vascular cell adhesion layer in the regions which the spacers contact. Therefore, when the spacers are rendered to have a pattern similar to that of the vascular cell adhesion portions, the vascular cell adhesive material only inside regions where no spacers are formed can be decomposed or denatured so that highly precise vascular cell adhesion-inhibiting portions can be formed. The use of the spacers also makes it possible that the active oxygen species generated by action of the photocatalyst reach the surface of the vascular cell adhesion layer, without diffusing, at a high concentration. Accordingly, highly precise vascular cell adhesion-inhibiting portion can be effectively formed.

In this embodiment, it is sufficient that such an arrangement state of the photocatalyst-containing layer side substrate is maintained only during the irradiation of energy.

The energy irradiation (exposure) mentioned in this embodiment is a concept that includes all energy ray irradiation that can decompose or denature the vascular cell adhesive material by the action of the photocatalyst upon irradiation with energy, and is not limited to light irradiation.

Normally, in such energy irradiation, ultraviolet light of 400 nm or less is used. This is because, as mentioned above, the photocatalyst that is preferably used as a photocatalyst is titanium dioxide, and as energy that activates a photocatalyst action by the titanium oxide, light having the above-mentioned wavelength is preferable.

As a light source that can be used in such energy irradiation, a mercury lamp, metal halide lamp, xenon lamp, excimer lamp and other various kinds of light sources can be cited.

Other than the method in which pattern irradiation is carried out via a photomask by using the above-mentioned light source, a method of carrying out drawing irradiation in a pattern by using laser such as excimer, YAG and the like can be applied. Furthermore, as mentioned above, when the base material has the light-shielding portion in a pattern same as that of the vascular cell adhesion portion, energy can be irradiated over the entire surface from the base material side. In this case, there are advantages in that there are no needs of the photomask and the like and a process of positional alignment and the like are also not necessary.

An amount of irradiation of energy at the energy irradiation is an amount of irradiation necessary for decomposing or denaturing the vascular cell adhesive material by the action of the photocatalyst.

At this time, by irradiating a layer containing the photocatalyst, with energy, while heating, the sensitivity can be raised; accordingly, it is preferable in that the vascular cell adhesive material can be efficiently decomposed or denatured. Specifically, it is preferable to heat in the range of 30° C. to 80° C.

The energy irradiation that is carried out via a photomask in this embodiment, when the above-mentioned base material is transparent, may be carried out from either direction of the base material side or a photocatalyst-containing layer side substrate. On the other hand, when the base material is opaque, it is necessary to irradiate energy from a photocatalyst-containing layer side substrate.

### Second Embodiment

In the second embodiment, at least a vascular cell adhesion-inhibiting layer, which inhibits adhesion to vascular cells and contains a vascular cell adhesion-inhibiting material decomposed or denatured by the action of a photocatalyst upon irradiation with energy, is formed on the base material, and in the above vascular cell adhesion portion, the vascular cell adhesion-inhibiting material is decomposed or denatured by the action of a photocatalyst upon irradiation with energy.

In this embodiment, the vascular cell adhesion-inhibiting material decomposed or denatured by the action of a photocatalyst upon irradiation with energy is contained in the vascular cell adhesion-inhibiting layer. Therefore, by arranging the vascular cell adhesion-inhibiting layer and the photocatalyst-containing layer so as to be opposite to each other and irradiating with energy in the pattern of the vascular cell adhesion portion, the vascular cell adhesion-inhibiting material in the vascular cell adhesion-inhibiting layer can be decomposed or denatured by the action of the photocatalyst in the photocatalyst-containing layer to form a vascular cell adhesion portion having adhesive properties to a vascular cell. Because the vascular cell adhesion-inhibiting material remains in the region not irradiated with energy, this region has no adhesive properties to a vascular cell and can be used as a vascular cell adhesion-inhibiting portion.

The phrase “the vascular cell adhesion-inhibiting material is decomposed or denatured” means that the vascular cell adhesion-inhibiting material is not contained, or that the vascular cell adhesion-inhibiting material is contained in a smaller amount than the amount of the vascular cell adhesion-inhibiting material contained in the vascular cell adhesion-inhibiting portion. For example, when the vascular cell adhesion-inhibiting material is decomposed by the action of a photocatalyst upon irradiation with energy, the vascular cell adhesion-inhibiting material is contained in a small amount in the vascular cell adhesion portion, or decomposed products etc. of the vascular cell adhesion-inhibiting material are contained, or the vascular cell adhesion-inhibiting material is completely decomposed to expose the base material. When the vascular cell adhesion-inhibiting material is denatured by the action of a photocatalyst upon irradiation with energy, its denatured products etc. are contained in the vascular cell adhesion portion. In this embodiment, the vascular cell adhesion portion preferably contains the vascular cell adhesive material having adhesive properties to a vascular cell, at least after irradiation with energy. The adhesive properties to a vascular cell of the vascular cell adhesion portion can thereby be increased, and vascular cells can be adhered highly accurately to the vascular cell adhesion portion only.

The surface distance of the vascular cell adhesion-inhibiting portion in this embodiment is usually about 200 μm to 1000 μm, particularly about 300 μm to 500 μm. Vascular cells can thereby be prevented from contacting with one another via pseudopods between the adjacent vascular cell adhesion portions.

The photocatalyst-containing layer side substrate, its arrangement, the energy irradiation method etc. used in this embodiment are the same as those described in the first embodiment described above, and thus their detailed description is omitted herein. Hereinafter, the vascular cell adhesion-inhibiting layer used in this embodiment is described.

The vascular cell adhesion-inhibiting layer used in this embodiment is not particularly limited insofar as it has vascular cell adhesion-inhibiting properties of inhibiting adhesion to vascular cells and contains a vascular cell adhesion-inhibiting material to be decomposed or denatured by the action of a photocatalyst upon irradiation with energy.

In this embodiment, the method for forming the layer is not particularly limited insofar as such layer can be formed. For example, the layer can be formed by coating a vascular cell adhesion-inhibiting layer-forming coating solution containing the vascular cell adhesion-inhibiting material, onto the photocatalyst-containing layer, by a common coating method. The thickness of the vascular cell adhesion-inhibiting layer can be suitably selected depending on the type etc. of the vascular cell culture patterning substrate, and can usually be about 0.01 μm to 1.0 μm, particularly about 0.1 μm to 0.3 μm.

The type etc. of the vascular cell adhesion-inhibiting material used in this embodiment are not particularly limited insofar as the vascular cell adhesion-inhibiting material has vascular cell adhesion-inhibiting properties of inhibiting adhesion to vascular cells and is decomposed or denatured by the action of a photocatalyst upon irradiation with energy.

The phrase “to have vascular cell adhesion-inhibiting properties” means to have a property of preventing vascular cells from being adhered to the vascular cell adhesion-inhibiting material, and when the adhesive properties to a vascular cell varies depending on the type of the vascular cell, the phrase means to have a property of inhibiting adhesion with the objective vascular cells.

The vascular cell adhesion-inhibiting material used in this embodiment is a material having such vascular cell adhesion-inhibiting properties. A material, which loses the vascular cell adhesion-inhibiting properties or which obtains good vascular cell adhesive properties, when decomposed or denatured by the action of a photocatalyst upon irradiation with energy is used.

As the vascular cell adhesion-inhibiting material, a material having high hydration ability can be used. The material having high hydration ability forms a hydration layer wherein water molecules gather around thereof. Usually, since such a material having high hydration ability has higher adhesion to water molecules than adhesion to vascular cells, the vascular cells cannot be adhered to the material having high hydration ability. Thus, the layer will have low adhesive properties to a vascular cell. The hydration ability is referred to as a property of hydrating with water molecules, and high hydration ability is intended to mean that the material is easily hydrated with water molecules.

As the material having high hydration ability which is used as a vascular cell adhesion-inhibiting material, for example, polyethylene glycol, amphoteric ionic materials having a betaine structure, phospholipid-containing materials, etc can be listed. When such materials are used as the vascular cell adhesion-inhibiting material, upon irradiated with energy in the below-described energy irradiating process, the vascular cell adhesion-inhibiting material is decomposed or denatured by the action of a photocatalyst so as to remove the hydration layer on the surface, thereby obtaining the material not having the vascular cell adhesion-inhibiting properties.

In this embodiment, a surfactant, which is decomposed by the action of a photocatalyst and has water repellent or oil repellent organic substituent, can also be used as the vascular cell adhesion-inhibiting material. As such surfactant for example, nonionic surfactants such as: hydrocarbon based such as the respective series of NIKKOL BL, BC, BO, and BB manufactured by Nikko Chemicals Co., Ltd.; and fluorine based or silicone based such as ZONYL FSN and FSO manufacture by Du Pont Kabushiki Kaisha, Surflon S-141 and 145 manufactured by ASAHI GLASS CO., LTD., Megaface F-141 and 144 manufactured by DAINIPPON INK AND CHEMICALS, Inc., FTERGENT F-200 and F251 manufactured by NEOS, UNIDYNE DS-401 and 402 manufactured by DAIKIN INDUSTRIES, Ltd., and Fluorad FC-170 and 176 manufactured by 3M can be cited. Also, cationic surfactants, anionic surfactants and amphoteric surfactants also can be used.

When the vascular cell adhesion-inhibiting layer is formed by using the above material as the vascular cell adhesion-inhibiting material, the vascular cell adhesion-inhibiting material is unevenly distributed on the surface. The water repellency or oil repellency can thereby be increased, and the interaction with vascular cells can be decreased to reduce adhesive properties to a vascular cell. Upon irradiation of this layer with energy in the energy irradiating process, the material is easily decomposed by the action of the photocatalyst to expose the photocatalyst. Thus, one not having the vascular cell adhesion-inhibiting properties can be obtained.

In this embodiment, a material, which obtains good adhesive properties to a vascular cell by the action of the photocatalyst upon irradiation with energy, is particularly preferably used as the vascular cell adhesion-inhibiting material. As such vascular cell adhesion-inhibiting material, for example, materials having oil repellency or water repellency can be listed.

When the material having oil repellency or water repellency is used as the vascular cell adhesion-inhibiting material, the interaction such as hydrophobic interaction between the vascular cells and the vascular cell adhesion-inhibiting material is made low by the water repellency or oil repellency of the vascular cell adhesion-inhibiting material, thereby decreasing adhesive properties to a vascular cell.

As the material having water repellency or oil repellency, a material, for example, which has such high bonding energy that the main skeleton thereof is not decomposed by the action of the photocatalyst and has water repellent or oil repellant organic substituent to be decomposed by action of the photocatalyst, can be listed.

Examples of such a material, which has such high bonding energy that the main skeleton thereof is not decomposed by the action of the photocatalyst and has water repellent or oil repellant organic substituent to be decomposed by action of the photocatalyst, include, for example, the materials used as the binder in the first embodiment, that is, (1) the organopolysiloxanes exhibiting high strength, obtained by hydrolyzing or polycondensating chloro- or alkoxysilanes by sol-gel reaction etc. and (2) organopolysiloxanes obtained by crosslinking reactive silicone.

When such material is used as the binder in the first embodiment, the material is used as a material having vascular cell adhesion-inhibiting properties by decomposing or denaturing the above-mentioned side chains of the organopolysiloxanes, in high ratio, so as to make it ultra-hydrophilic by the action of the photocatalyst upon irradiation with energy. However, in this embodiment, the region irradiated with the energy can have adhesive properties to a vascular cell by irradiating with energy to such a degree that side chains of the organopolysiloxanes are not completely decomposed or denatured by the action of the photocatalyst upon irradiation with energy. Together with the above-mentioned organopolysiloxane, a stable organosilicon compound not undergoing any crosslinking reaction, such as dimethylpolysiloxane, can also be separately mixed.

When the material having water repellency or oil repellency is used as the vascular cell adhesion-inhibiting material, the material preferably has a contact angle, with water, of 80° or more, particularly in the range of 100° to 130°. With this contact angle given, the adhesive properties to a vascular cell, of the vascular cell adhesion-inhibiting layer before irradiation with energy can be reduced. The upper limit of the angle is the upper limit of the contact angle, with water, of the vascular cell adhesion-inhibiting material on a flat base material. For example, when the contact angle, with water, of the vascular cell adhesion-inhibiting material on a base material with concavoconvex is measured, the upper limit may be about 160° as shown by Ogawa et al. in Japanese Journal of Applied Physics, Part 2, Vol. 32, L614-L615, 1993.

When this vascular cell adhesion-inhibiting material is irradiated with energy to impart the adhesive properties to a vascular cell, the material is preferably irradiated with energy such that the contact angle thereof with water comes to be in the range of 10° to 40°, particularly 15° to 30°. The adhesive properties to a vascular cell of the vascular cell adhesion-inhibiting layer after energy irradiation can thereby be increased. The contact angle with water can be obtained by the method described above.

The vascular cell adhesion-inhibiting material is contained preferably in the range of 0.01% by weight to 95% by weight, particularly 1% by weight to 10% by weight, in the vascular cell adhesion-inhibiting layer. The region containing the vascular cell adhesion-inhibiting material can thereby be a region of low adhesive properties to a vascular cell.

The vascular cell adhesion-inhibiting material preferably has surface activity. For example, when drying the vascular cell adhesion-inhibiting layer-forming coating solution or the like containing the vascular cell adhesion-inhibiting material after coating thereof, the material is distributed highly unevenly on the surface of the coating film, thus giving excellent vascular cell adhesion-inhibiting properties.

The vascular cell adhesion-inhibiting layer in this embodiment may contain a binder and the like in accordance with required characteristics such as coating properties in formation of the layer, strength and resistance of the formed layer. The vascular cell adhesion-inhibiting material may also function as the binder.

As the binder, for example, a binder having such high bonding energy that its main skeleton is not decomposed by the action of the photocatalyst can be used. Specific examples of the binder include polysiloxane etc. not having organic substituents or having organic substituents to such a degree that adhesive properties is not adversely affected, and such polysiloxane can be obtained by hydrolyzing or polycondensating tetramethoxysilane, tetraethoxysilane etc.

In this embodiment, the binder is contained preferably in the range of 5% by weight to 95% by weight, more preferably 40% by weight to 90% by weight, still more preferably 60% by weight to 80% by weight, in the vascular cell adhesion-inhibiting layer. By incorporation of the binder in this range, formation of the vascular cell adhesion-inhibiting layer can be facilitated and the vascular cell adhesion-inhibiting layer can be endowed with strength etc. thus allowing it to exhibit its characteristics.

In this embodiment, the vascular cell adhesion-inhibiting layer preferably contains a vascular cell adhesive material having adhesive properties to a vascular cell, at least after irradiation with energy. By this, in the vascular cell adhesion-inhibiting layer, adhesive properties to a vascular cell of the vascular cell adhesion portion, which is the region irradiated with energy, can be improved. The vascular cell adhesive material may be a material usable as the binder or may be a material used separately from the binder. The vascular cell adhesive material may have good adhesive properties to a vascular cell prior to irradiation with energy, or may be endowed with good adhesive properties to a vascular cell by the action of the photocatalyst upon irradiation with energy. The wording “having adhesive properties to a vascular cell” refers to good adhesion to vascular cells, and when the adhesive properties to a vascular cell vary depending on the type of vascular cell, the wording refers to good adhesion to the objective vascular cells.

In this embodiment, as long as the vascular cell adhesive material have good adhesive properties to a vascular cell at least after being irradiated with energy. For example, the adhesive properties to a vascular cell can be improved by biological characteristics or by physical interaction such as hydrophobic interaction, electrostatic interaction, hydrogen bonding, van der Waals force.

In this embodiment, the vascular cell adhesive material is contained preferably in the range of 0.01% by weight to 95% by weight, particularly 1% by weight to 10% by weight, in the vascular cell adhesion-inhibiting layer. By this, the vascular cell adhesion-inhibiting layer can further improve the adhesive properties to a vascular cell of the vascular cell adhesion portion, which is a region irradiated with energy. When the material having good adhesive properties to a vascular cell prior to irradiation with energy is used as the vascular cell adhesive material, the material is preferably contained to such a degree as not to inhibit the vascular cell adhesion-inhibiting properties of the vascular cell adhesion-inhibiting material in the region not irradiated with energy, that is, the region serving as the vascular cell adhesion-inhibiting portion.

### B. Method for Manufacturing Blood Vessel

Now, the method for manufacturing a blood vessel according to the present invention is described. The method for manufacturing blood vessels according to the present invention is a method which comprises culturing vascular cells with the above-described vascular cell culture patterning substrate to manufacture a blood vessel.

In the present invention, vascular cells are cultured by using the above-described vascular cell culture patterning substrate, whereby the vascular cells can be cultured into an objective pattern in high precision, without the vascular cells on the adjacent vascular cell adhesion portions being contacted to one another, during culturing, via generated pseudopods. Thus, the formed blood vessels will not be ruptured so that the high quality blood vessels can be obtained.

The vascular cell culture patterning substrate is the same as described, and thus its detailed description is omitted herein, and the vascular cells used in the present invention are described.

The vascular cells used in the present invention are vascular cells which form a blood vessel by being cultured. It refers to vascular endothelial cells, pericytes, smooth muscle cells, endothelial precursor cells and smooth muscle precursor cells derived from organisms, particularly animals. Particularly, it refers to vascular endothelial cells etc. Plural kinds of cells can be co-cultured such as co-culture of vascular endothelial cells and pericytes or co-culture of endothelial cells and smooth muscle cells.

To form blood vessels, when culturing the vascular cells by adhering them on the vascular cell adhesion portion, it is effective to apply shearing stress in uniaxial direction in the same direction as the line pattern of the vascular cell adhesion portion. The adhered form of the vascular cells can thereby become long and thin spindle-shaped, and the respective vascular cells can adhere to one another in such a state that they seem oriented in the uniaxial direction described above. To form blood vessels, it is important that the vascular cells are adhered in a confluent state such that the vascular cells are adhered in a thin and long form and the vascular cells are directed to the same direction. The method for applying shear stress in the uniaxial direction includes: a method in which the vascular cells are cultured by placing a culture dish on a shaker or a shaking apparatus; and a method in which the vascular cells are cultured while streaming culture liquid in one direction. To forma blood vessel of 5000 μm or more in width, shearing stress in uniaxial direction is essential.

Usually, a blood vessel is obtained by forming the vascular cells in an objective pattern on the vascular cell adhesion portion, and then, adding, to a medium, growth factors such as bFGF and VEGF promoting vascularization of vascular cells. It is estimated that, by stimulation from the growth factors, proliferation of the vascular cells is terminated and differentiated so as to be blood vessels. As the medium for vascularization of vascular cells adhered in a confluent state to the vascular cell adhesion portion, not only a liquid medium containing the growth factor, but also a gelled medium containing the above-described growth factor or a combination of gelled and liquid mediums containing the growth factor can be used. As the gelled medium, collagen, fibrin gel, Matrigel (trade name) or synthetic peptide hydrogel can be used.

The present invention is not limited to the above mentioned embodiments. The above mentioned embodiments are merely examples, and any one having the substantially same configuration and the same effects, or equivalent thereof, as the technological idea disclosed in the claims of the present invention is included in the technological scope of the present invention.

### EXAMPLES

Hereinafter, the present invention will be more specifically described by referring to the Examples.

### Example 1

### (Formation of a Photomask Having a Photocatalyst-Containing Layer)

A quartz photomask having a stripe pattern of 40 μm glass openings and 500 μm metal light-shielding portions was prepared. Subsequently, 5 g of trimethoxymethylsilane TSL8114 (GE Toshiba Silicones) and 2.5 g of 0.5 N hydrochloric acid were mixed and stirred for 8 hours. The mixture was diluted 10-fold with isopropyl alcohol to prepare a primer layer composition. This primer layer composition was coated onto the patterned surface of the photomask by spin coating, and the substrate was dried at a temperature of 150° C. for 10 minutes to form a photomask provided with a primer layer.

Then, 30 g of isopropyl alcohol, 3 g of trimethoxymethylsilane TSL8114 (GE Toshiba Silicones), and 20 g of a photocatalyst inorganic coating agent ST-K03 (ISHIHARA SANGYOKAISYA, LTD.) were mixed and stirred at 100° C. for 20 minutes. The mixture was diluted 3-fold with isopropyl alcohol to prepare a photocatalyst-containing layer composition.

This photocatalyst-containing layer composition was coated, by spin coating, onto the photomask substrate provided with the primer layer, and then, dried at 150° C. for 10 minutes to form a photomask having a transparent photocatalyst-containing layer.

### (Formation of a Vascular Cell Adhesion-Inhibiting Layer)

Five (5.0) grams of organosilane TSL-8114 (Toshiba Silicones), 1.5 g of fluoroalkylsilane TSL-8233 (Toshiba Silicones) and 2.36 g of 0.005 N hydrochloric acid were mixed and stirred for 24 hours. This solution was diluted 100-fold with isopropyl alcohol and coated by spin coating onto a soda glass substrate previously subjected to alkali treatment, and the substrate was dried at a temperature of 150° C. for 10 minutes to allow hydrolysis and polycondensation reaction to advance to give a patterning substrate having a vascular cell adhesion-inhibiting layer of 0.2 μm in thickness.

### (Formation of the Vascular Cell Culture Patterning Substrate)

The vascular cell adhesion-inhibiting layer of this patterning substrate was opposed to the photocatalyst-containing layer, containing a photocatalyst, of the photomask. Then, the above was exposed via the photomask to ultraviolet rays, with 6 J/cm² energy, from a mercury lamp. Thereby, a vascular cell culture patterning substrate having a vascular cell adhesive surface patterned, such that the unexposed portions having vascular cell adhesion-inhibiting properties and the exposed portions having vascular cell adhesive properties, was obtained. Then, the vascular cell culture patterning culture substrate was cut into a specimen with a size of 15 mm×25 mm. In this cutting, the specimen was cut such that the line pattern of the vascular cell adhesion portion corresponds to the major axis of the vascular cell culture patterning culture substrate.

### (Dissemination of Cells and Formation of Tissue)

The substrate was dipped in DMEM medium containing 10% bovine fetal serum, and primary human umbilical vein endothelial cells (HUVECs) were disseminated so as to be a concentration of 2×10⁵ cells/ml. The cells were cultured at 37° C. in a 5% carbon dioxide atmosphere for 24 hours to allow the vascular cells to adhere to the vascular cell adhesion portion.

When the vascular cells that had adhered to the substrate were observed, it was confirmed that the vascular cells were aligned along all region in the vascular cell culture region, the vascular cells were in an extended form, and there is no contacting of the pseudopods between the vascular cell adhesion portions.

Further, the DMEM medium was exchanged with one containing bFGF (Sigma) at a concentration of 10 ng/ml, culturing was continued at 37° C. in a 5% carbon dioxide atmosphere for 24 hours, and formation of a vascular tissue composed of continuous vascular cells was confirmed.

### Comparative Example 1

An experiment was carried out in the same manner as in Example 1 except that the photomask was exchanged to one having a stripe pattern with 40 μm vascular cell adhesion portions/150 μm vascular cell adhesion-inhibiting portions. As a result, it was confirmed that, after 24 hours of culturing, the vascular cells had adhered, in an extended form, to the vascular cell adhesion portion, but pseudopods generated from the vascular cells had extended to a part of the vascular cell adhesion-inhibiting portion.

Further, bFGF was added to the DMEM medium similarly to Example 1 to form a tissue of the vascular cells, and a blood vessel was formed. However, it was confirmed that, as compared with Example 1, the blood vessel was ruptured at some places, the length was shorter, also, adjacent blood vessels were in contact to one another, and formation of the tissue was incomplete.

### Example 2

A substrate was prepared in the same manner as in Comparative Example 1, and a polytetrafluoroethylene plate having a height width of 50 μm and a height of 5 μm was stuck on the center of the vascular cell adhesion-inhibiting portion of the substrate. Vascular cells were disseminated onto this substrate in the same manner as in Example 1.

By observing the vascular cells that had adhered to the substrate, it was confirmed that the vascular cells were oriented in the direction along the whole region in the vascular cell adhesion portion, showed an extended form, and were not contacted with one another via pseudopods between the vascular cell adhesion portions.

After the DMEM medium was exchanged with one containing bFGF (Sigma) at a concentration of 10 ng/ml, the cells were cultured at 37° C. in a 5% carbon dioxide atmosphere for 24 hours, and formation of a vascular tissue composed of continuous vascular cells was confirmed.

### Example 3

### (Formation of a Photomask for Forming a Vascular Cell Adhesion-Inhibiting Portion)

A 5-inch quartz photomask having an alignment mark, with openings of 500 μm in width and light-shielding portions of 200 μm in width, was prepared.

### (Formation of a Photomask Having a Photocatalyst-Containing Layer Having a Vascular Cell Adhesion Auxiliary Portion)

A 5-inch quartz photomask comprising: an alignment mark corresponding to the alignment mark of the above photomask; a light-shielding portion corresponding to the opening lines of the above photomask; and an opening pattern for a vascular cell adhesion portion having a vascular cell adhesion auxiliary portion, formed along the line at a position corresponding to the light-shielding portion lines of the photomask, was prepared. The opening pattern was formed into a pattern having light-shielding portions of 4.5 μm in width and openings of 35.5 μm in width, and the light-shielding pattern served as the pattern of the vascular cell adhesion auxiliary portion. Subsequently, a photocatalyst-containing layer was formed on this photomask by the same method as in Example 1.

### (Formation of a Vascular Cell Adhesion-Inhibiting Portion)

1% Polymerization initiator 2,2-dimethoxy-2-phenylacetophenone was dissolved in polyethylene glycol diacrylate (Aldrich). This solution was coated by spin coating onto a glass substrate previously surface-treated by dipping for 2 hours in a dehydrated toluene solution (concentration 3%) of γ-methacryloxypropyltrimethoxysilane (GE Toshiba Silicones). Subsequently, the substrate was subjected to a UV-light exposure step with the above vascular cell adhesion-inhibiting portion-forming photomask and then to a water development step to form a convex vascular cell adhesion-inhibiting portion of 500 μm in width and 0.8 μm in thickness composed of polyethylene glycol gel.

### (Formation of a Vascular Cell Adhesion Portion Having a Vascular Cell Adhesion Auxiliary Portion)

A silane coupling agent XC98-B2472 (GE Toshiba Silicones), having vascular cell adhesion-inhibiting properties and also exhibits vascular cell adhesive properties when being decomposed by the action of a photocatalyst upon irradiation with energy, was diluted 10-fold with isopropyl alcohol, and 1,3-butanediol to a concentration of 10% was added and stirred to prepare a coating agent for forming a vascular cell adhesion portion and a vascular cell adhesion auxiliary portion. The glass substrate having the convex vascular cell adhesion-inhibiting portion was cleaned with UV for 120 seconds, and immediately thereafter, the coating agent was coated thereto by spin coating and dried at 60° C. for 24 hours. Thereafter, the substrate was washed well with water and dried again.

The pattern of the vascular cell adhesion auxiliary portion, and the photomask having a photocatalyst-containing layer, were arranged by positioning with the alignment mark such that the photocatalyst-containing layer was opposite to the substrate surface having the convex vascular cell adhesion-inhibiting portion. Then, the backside of the photomask with the photocatalyst-containing layer was irradiated with 6 J/cm² UV light. The vascular cell adhesion portion having the vascular cell adhesion auxiliary portion was thereby prepared. This substrate was cut into pieces with a size of 15 mm×25 mm in the same manner as in Example 1.

### [Dissemination of Vascular Cells and Formation of Tissue]

The substrate was arranged on a culture dish, and HUVECs were disseminated at a concentration of 6×10⁵ cells/ml. The culture dish was arranged on a seesaw shaker and the vascular cells were cultured for 24 hours in the same manner as in Example 1, to allow the vascular cells to adhere to the vascular cell adhesion portion having the vascular cell adhesion auxiliary portion. While culturing, the shaker was operated as a seesaw to allow the medium to flow in the same direction as in the line pattern of the substrate.

After culturing for 24 hours, the medium was carefully removed by suction, and then, 0.5 ml Matrigel (Becton Dickinson) containing bFGF (Sigma) at a concentration of 10 ng/ml was given, as a new medium, onto the substrate. The above was gelled, DMEM medium containing 0.5% fetal bovine serum was added and was cultured. The cells were cultured at 37° C. in an atmosphere of 5% carbon dioxide for 24 hours, and it was confirmed that the vascular cells had formed a continuous vascular tissue.

1. A vascular cell culture patterning substrate comprising: a base material; a vascular cell adhesion portion formed in at least two substantially parallel lines on the base material, and has an adhesive properties to a vascular cell which forms a blood vessel; and a vascular cell adhesion-inhibiting portion formed in between two adjacent vascular cell adhesion portions on the base material, and inhibits adhesion to the vascular cell,
wherein the vascular cell adhesion-inhibiting portions are formed at such a surface distance that, upon adhesion of the vascular cells to the vascular cell adhesion portions, the cells on the two vascular cell adhesion portions adjacent to the vascular cell adhesion-inhibiting portion are not contacted with one another via pseudopods.

2. The vascular cell culture patterning substrate according to claim 1, wherein the vascular cell adhesion-inhibiting portion is formed in a convex form.

3. A method for manufacturing a blood vessel, wherein the vascular cell culture patterning substrate according to claim 1 is used to culture a vascular cell.

4. A method for manufacturing a blood vessel, wherein the vascular cell culture patterning substrate according to claim 2 is used to culture a vascular cell.
